(19) United States
(12) Patent Application Publication
(10) Pub. No.: US 2012/0203132 A1
(43) Pub. Date: Aug. 9, 2012

(54) DEVICE AND METHOD FOR APPLYING PRESSURE TO A BODY LIMB

(75) Inventors: Uzi Blumensohn, Yahoud (IL); Yair Galili, Or Yehuda (IL)

(73) Assignee: GBVEINTECH LTD., Yahoud (IL)

(21) Appl. No.: 13/379,237

(22) PCT Filed: Jun. 20, 2010
(86) PCT No.: PCT/IL10/00484

§ 371 Date: Apr. 24, 2012

Related U.S. Application Data
(60) Provisional application No. 61/213,565, filed on Jun. 19, 2009.

(51) Int. Cl. A61B 5/04

(52) U.S. Cl. 600/546

(57) ABSTRACT

Provided is a device for providing pressure to a body limb. The device includes an actuating unit configured to apply a varying pressure to a first region of the limb and a sensing unit monitoring a state of a second region of the limb and generating a time dependent signal indicative of the state of the second region of the limb. A processor analyzes the signal generated by the sensing unit and determines a time dependent state of the second region. The processor activates the actuating unit to generate a time dependent pressure on the first region of the body limb based upon the time dependent state of the second region.

### FIELD OF THE INVENTION

This invention relates to medical devices for applying pressure to a body limb.

### BACKGROUND OF THE INVENTION

Many vessels in animals transport fluids from one body location to another in a substantially unidirectional manner along the length of the vessel. Veins in the body transport blood to the heart while arteries carry blood away from the heart. The veins, mainly those in the legs, have one-way valve structures in the form of leaflets disposed annularly along the inside wall of the veins which periodically open to permit blood flow towards the heart and then close to prevent back-flow

In many mammals, small semilunar valves known as “venous valves” are found within the veins of the extremities. Such venous valves function as one-way check valves to maintain the flow of venous return blood in the direction toward the heart, while preventing blood from back-flowing away from the heart. When blood flows through the vein, the pressure forces the valve leaflets apart as they flex in the direction of blood flow and move towards the inner wall of the vessel, creating an opening for blood flow. The leaflets, however, do not normally bend in the opposite direction and therefore return to a closed position to prevent blood flow in the opposite, i.e. retrograde, direction after the pressure is relieved. The leaflet structures, when functioning properly, extend radially inwardly toward one another such that the tips contact each other to block backflow of blood.

Heart valves open and close 60 to 150 times per minute with pressures of up to 250 mm Hg. On the other hand, venous valves typically remain open with minimal forward flow and close with flow reversal. Reverse venous flow may develop intermittent pressures of 150 mm Hg. Venous valves are particularly important in the veins of the lower extremities, as venous blood returning from the lower extremities is required to move against a long hydrostatic column, especially when the individual is in a standing or upright position. Venous valves are typically bicuspid valves positioned at varying intervals along the veins to permit substantially unidirectional blood flow toward the heart. These natural venous valves open to permit the flow of fluid in the desired direction, and close upon a change in pressure, such as a transition from relaxation to activation of shin muscles.

Venous valves, especially those in the upper leg, perform an important function. When a person rises from a seated to a standing position, arterial blood pressure increases instantaneously to insure adequate perfusion to the brain and other critical organs. In the legs and arms, the transit time of this increased arterial pressure is delayed, resulting in a temporary drop in venous pressure. The venous pressure in the feet of someone walking is of the order of 25 mmHg (3.3 kPa), whereas in the feet of an individual standing absolutely still it is of the order of 90 mmHg (12 kPa). Venous pressure drops as blood flow responds to body position change and gravity, thereby reducing the volume of blood available to the right heart and possibly reducing the flow of oxygenated blood to the brain. In such a case, a person could become light headed, dizzy or experience syncope. It is the function of valves in the iliac, femoral and the more distal veins, to detect these drops in pressure and resulting change of direction of blood flow and to close in order to prevent blood from pooling in the legs so as to maintain blood volume in the heart and head. The valves reopen and the system returns to normal forward flow when the reflected arterial pressure again appears in the venous circulation. Compromised valves, however, would allow reverse blood flow and pooling.

Occasionally, congenital defects or injury to valves within a body vessel can result in an undesirable amount of retrograde fluid flow across a valve, and compromise the unidirectional flow of fluid across the valve. In the condition of venous valve insufficiency, the valve leaflets thicken and lose flexibility resulting in the inability of the valves to extend sufficiently radially inward to enable their tips to come into sufficient contact with each other to prevent retrograde blood flow. If left untreated, venous valve insufficiency can result in excessive retrograde venous blood flow through incompetent venous valve. The retrograde blood flow causes the buildup of hydrostatic pressure on the residual valves and the weight of the blood dilates the wall of the vessel. Such retrograde blood flow, commonly referred to as reflux, leads to swelling and varicose veins, causing great discomfort and pain to the patient. Such retrograde blood flow, if left untreated can also cause venous stasis ulcers of the skin and subcutaneous tissue. There are generally two types of venous valve insufficiency: primary and secondary. Primary venous valve insufficiency is typically a congenital condition in which the vein is too wide in relation to the leaflets so that the leaflets cannot come into adequate contact to prevent backflow. More common is secondary venous valve insufficiency which is caused by clots which gel and scar, thereby changing the configuration of the leaflets, i.e. thickening the leaflets creating a “stub-like” configuration. Venous valve insufficiency can occur in the superficial venous system, such as the saphenous veins in the leg, or in the deep venous system, such as the femoral and popliteal veins extending along the back of the knee to the groin. Chronic venous insufficiency (CVI) arises from long duration venous hypertension caused by valvular insufficiency and/or venous obstruction secondary to venous thrombosis. Other primary causes of CVI include varicosities of long duration, venous hypoplasia and arteriovenous fistula. The signs and symptoms of CVI have been used to classify the degree of severity of the disease, and reporting standards have been published. Studies demonstrate that deterioration of venous hemodynamic status correlates with disease severity. Venous reflux, measured by ultrasound studies, is the method of choice of initial evaluation of patients with pain and/or swelling in the lower extremities. In most serious cases of CVI, venous stasis ulcers are indicative of incompetent venous valves which can occur in superficial, common, deep or communicating veins.

While the etiology and pathophysiology of CVI and resulting venous ulcers and other symptoms and signs are well established, there has not been satisfactory progress in the treatment of this problem. A common method of treatment of venous valve insufficiency is placement of an elastic stocking around the patient's leg to apply external pressure to the vein, forcing the walls radially inwardly to force the leaflets into apposition and reducing edema formation. However, it was found that the elastic stockings do not exert sufficient pressure to reduce venous reflux, although they help many patients by reducing discomfort.

Moreover, the tight stocking is quite uncomfortable and sometimes even painful, especially in warm weather, or in the case of tender and ulcerated legs which are typical in CVI patients, as the stocking must be constantly worn during the day. Furthermore, old and debilitated people have difficulties in applying the socking due to the posture and physical strength needed to put it on. The elastic stocking also affects the patient's physical appearance, thereby potentially having an adverse psychological effect. This physical and/or psychological discomfort often results poor patient compliance. Above all, although elastic stockings sometimes help to reduce symptoms like edema and swelling, they cannot give a full and definitive solution to CVI and its sever complications such as stasis ulcers.

Another device for aiding cardiocepital venous flow to prevent venous hypertension resulting from reflux is a boot placed around the foot and leg that apples pressure to the foot and leg. However, the boot is cumbersome and usually requires the patient to remain immobile, and thus cannot be useful and effective for an ambulatory patient during regular daily activities. The boot enhances venous flow toward the heart but does not reduce venous reflux which is the principal malfunction in CVI. Thus, use of a boot increases the normal function of the legs i.e the pumping calf muscle activity, but does not correct the basic and fundamental malfunction of chronic venous insufficiency which is the malfunction of the one way valves located in the veins.

A few surgical interventions have been developed to avoid the discomfort and inefficiency of the stocking and inflatable boots. These methods include major surgery requiring the application of a cuff directly around the vein in order to reduce its diameter and to approximate the valve leaflets. This surgery requires a large incision, resulting in a long patient recovery time, scarring, and carries the inherent risks of surgery. This surgical intervention is only appropriate patients with non damaged valve leaflets. Thus, many patients with damaged leaflets are not candidates for this procedure.

Another invasive method of surgery involves selective repairing of the valve leaflets, referred to as valvuloplasty. In this method, sutures are utilized to bring the free edges of the valve cusp into contact. This procedure is complicated and also has the inherent risks of surgery. Another surgical method is reimplantation of a vein section which contains a normally functioning valve harvested from another part of the body into the incompetent vein. Invasive operations have not gained popularity due to their complexity and high complication rates.

Short term stretch bandages applied to the thigh capable of exerting a pressure of 40 to 60 mm Hg to the thigh have been examined. This measure has been found to provide considerable hemodynamic improvement, including reduced venous reflux and reduce venous pressure in the lower leg, even in patients with severe chronic venous insufficiency from deep vein incompetence. [Partsch et. al, Journal of vascular surgery 2002, vol. 36, no 5, pp. 948-952]

During standing without moving, the hydrostatic pressure in the vein increases and reaches gradually to over 80 mm-Hg at ankle level and about 50 mm-hg at the belt level 5 cm below knee in both normal and CVI limbs. However, during foot and shin movements as in walking, there is a significant difference in the so-called ambulatory venous pressure (AVP) between a normal and diseased limb. In the normal limb, the ankle vein pressure is 30 mm Hg and less, while in the CVI limb the pressure is more than twofold higher (above 80 mm Hg).

### SUMMARY OF THE INVENTION

The present invention provides a device and method for providing pressure to a body limb such as a leg. Use of the device may be used in the treatment of CVI complications such as edema and stasis ulcers.

In accordance with the invention, the state of the limb is continuously monitored and a varying amount of pressure is applied to the limb according to the current state of the limb muscles. The device of the invention thus synchronizes the pressure applied to the limb with the state of the limb muscles, and tends to modulate blood flow in the limb to provide a greater resistance to fluid flow through the body vessel in a retrograde direction (i.e., away from the heart) than in an antegrade direction (i.e., toward the heart).

The device of the invention comprises an actuating unit, a sensing unit and a control unit. The actuating unit is under the control of the control unit and is configured to apply a radially inward pressure on a first region of the limb. The pressure may be applied, for example, by varying the effective length of an inelastic band or ring surrounding the first region of the limb. Alternatively, the pressure may be applied by inflating an inflatable sleeve surrounding the limb. The sensing unit monitors the circumference of a second region of the limb and transmits a time dependent signal indicative of the calf circumference to the control unit. The control unit analyzes the signal received from the sensing unit and determines a state of the limb. The state of the limb may be, for example, that the limb is resting, the limb is in muscle vein pump diastole (the limb muscles are relaxed), or that the limb is in muscle vein pump systole (the limb muscles are squeezing the inner deep veins). When the limb is in muscle vein pump systole, the processor activates the actuating unit to decrease the pressure on the first region of the limb, so that the pressure from the deep vein is released at the first region. When the limb is in muscle vein pump diastole, the processor activates the actuating unit to increase the pressure on the first region of the limb to reduce or prevent retrograde blood flow.

During walking, episodes of muscle vein pump systole alternate with episodes of muscle vein pump diastole, so that during walking, the device provides a periodically fluctuating pressure to the limb that is synchronized with the state of the muscle vein pump.

Thus, in its first aspect the invention provides a device for providing pressure to a body limb comprising:
(a) an actuating unit configured to apply a varying pressure to a first region of the limb;
(b) a sensing unit monitoring a state of a second region of the limb and generating a time dependent signal indicative of the state of the second region of the limb; and
(c) a control unit comprising a processor configured to:
   receive the signal generated by the sensing unit;
   analyze the signal to determine a time dependent state of the second region; and
   activate the actuating region to generate a time dependent pressure on the first region of the body limb based upon the time dependent state of the second region.

The sensing unit may sense a circumference of the second region of the limb. In this case, the sensing unit may comprise an elastic band and a transducer monitoring an extent of stretching of the elastic band. The sensing unit may sense electrical activity of a muscle in the limb, for example, using surface electrodes placed on the skin surface over the limb muscle.

The the actuating unit may comprise a band or ring configured to surround the first region of the limb. The actuating unit may comprise an inflatable ring configured to surround the first region of the limb.

A state of the second region of the limb may be that the second region of the limb is any one or more of the limb is resting, the limb is moving the limb is in muscle vein pump diastole, and the limb is in muscle vein pump systole

The processor may be configured to activate the actuating unit to decrease the pressure on the first region of the limb when the limb is in muscle vein pump systole. The processor may be configured to activate the actuating unit to increase the pressure on the first region of the limb when the limb is in muscle vein pump diastole.

In its second aspect, the invention provides a method for providing pressure to a body limb comprising:
(a) monitoring a state of a second region of the limb;
(b) generating a time dependent pressure on a first region of the body limb based upon the time dependent state of the second region.

### BRIEF DESCRIPTION OF THE DRAWINGS

In order to understand the invention and to see how it may be carried out in practice, embodiments will now be described, by way of non-limiting example only, with reference to the accompanying drawings, in which:

FIG. 1 shows a device for applying pressure to a body limb in accordance with one embodiment of the invention; and

FIG. 2 shows the actuating unit of the device of Fig. in greater detail; and

FIG. 3 shows modulation of limb pressure exerted by the device of the invention during walking

### DETAILED DESCRIPTION OF EMBODIMENTS

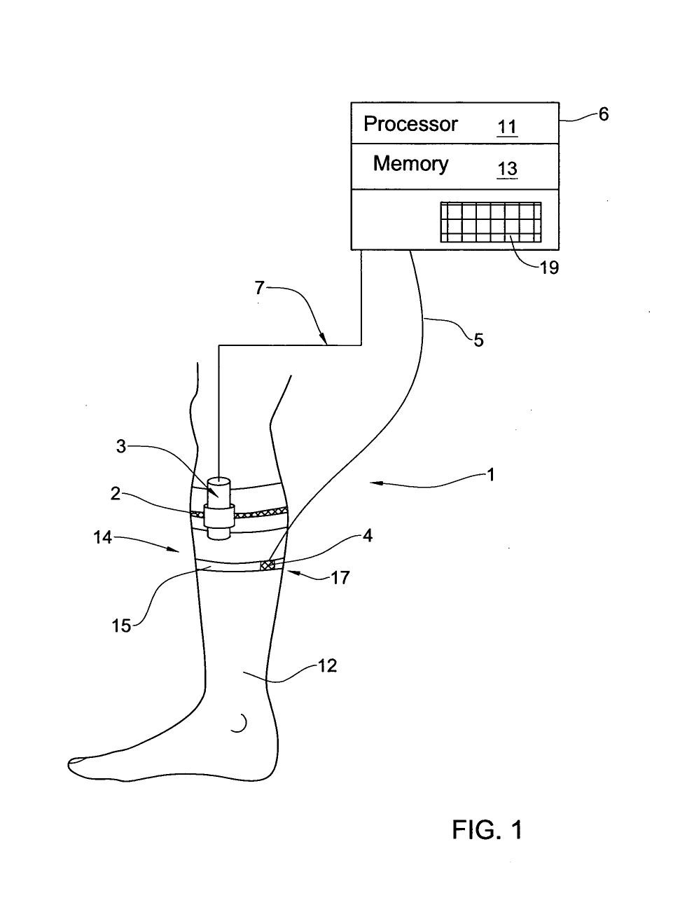

FIG. 1 shows a device 1 for applying pressure to a body limb 12, such as a leg, in accordance with one embodiment of the invention. The device 1 has includes an actuating unit 14, a sensing unit 17 and a control unit 6. The actuating unit 14 includes a flexible inelastic band 2 that may be, for example, about 15 mm wide. The actuating unit 14 is configured to encircle the limb 12 at a first region of the limb, for example, the shin region of a leg, and an electric motor 3. Activation of the electric motor 3 is under the control of the control unit 6, as explained in detail below. Activation of the motor 3 alters the effective length of the band 2 in order to apply a variable pressure to the leg 12. The sensing unit 17 includes a band 15 configured to surround the limb 12 at a second region of the limb, for example, the calf region. A transducer 4 associated with the band 15 monitors a force on the band 15 which is indicative of the circumference of the calf region and generates a time dependent signal indicative of the calf circumference. The signal is transmitted to the control unit 6. The control unit comprises a processor 11, a memory 13 and a user keyboard 19. The control unit may be carried in a pouch mounted on the user's belt. Each of the actuating unit, the sensing unit, and the processor is provided with a power source (not shown) which is preferably a rechargeable battery.

Communication between the transducer 4 and the control unit 6 may be via a wired connection 5 or by a wireless connection. The processor monitors the signal and determines a state of the limb. The state of the limb may be, for example, that the limb is resting, the limb is moving, the limb is in muscle vein pump diastole (e.g. the lower leg muscles are relaxed), or that the limb is in muscle vein pump systole (e.g. the calf muscles are squeezing the inner deep veins producing an elevated pressure and causing a blood flow upwards toward the heart).

Another embodiment of the invention (not shown) determines the sensing unit determines a state of the limb by monitoring electrical activity of limb muscle, for example, using surface electrodes placed on the skin surface over the limb muscle.

The processor communicates with the motor 3 via a communication line 7 in order to adjust the effective length of the band 2 according to the determined state of the limb in order to reduce AVP in the limb. When the limb is in muscle vein pump systole, the processor activates the motor 3 to increase the circumference of the band 2 thus decreasing the pressure on the shin, so that the pressure from the deep vein is released below the knee, the vein expands, and blood can flow upward towards the heart. When the limb is in muscle vein pump diastole, the processor activates the motor 3 to decrease the circumference of the band 2 which compresses the vein in a way that reduces or prevents retrograde blood flow.

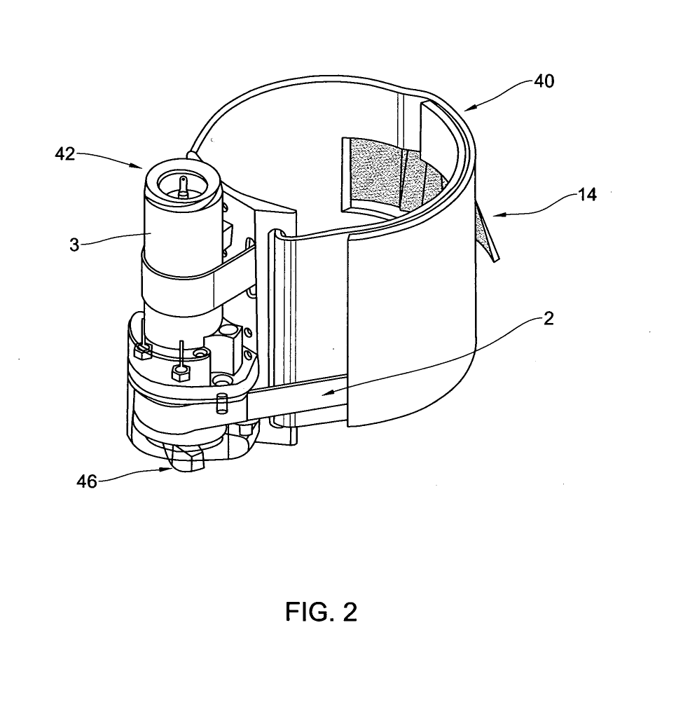

FIG. 2 shows the actuating unit 14 in greater detail. An adjustable holding belt 40 allows mounting of the actuating unit 14 to the first region of the body limb. The band 2 is located inside or on the holding belt 40. A tensioning actuator 42 allows setting a desired baseline pressure to the band 2. A release knob 46 allows manual release of the band 2 when required.

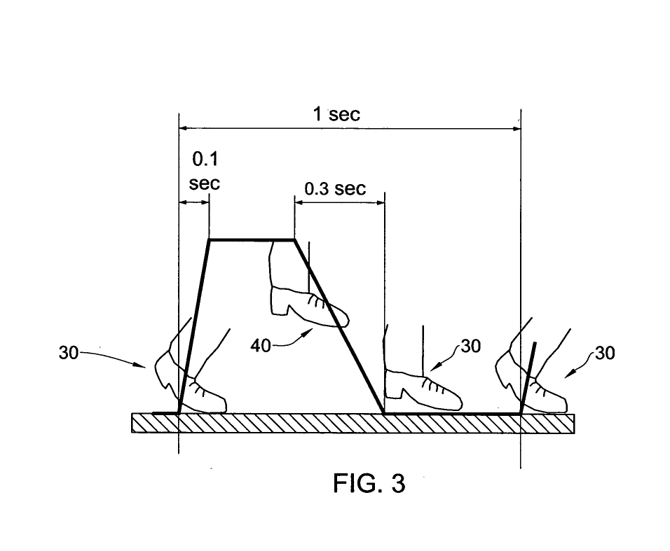

During walking, episodes of muscle vein pump systole alternate with episodes of muscle vein pump diastole, so that during walking, the device 1 provides a periodically fluctuating pressure to the limb that is synchronized with the state of the muscle vein pump. FIG. 3 shows a graph showing the pressure applied to the shin region of a leg by the actuating unit during walking, in accordance with one embodiment of the invention. For a walking cycle of about 1 sec duration, when the leg is in muscle vein pump systole 30, the processor activates the actuating unit 14 to gradually relax the shin region over a period of about 0.1 sec to achieve a reduced pressure on the shin region that is maintained for about 0.3 sec and which tends to allow antegrade blood flow toward the heart. When the leg is in muscle vein pump diastole 40, the processor activates the actuating unit to gradually increase the pressure on the shin over a period of about 0.3 sec that is maintained for about another 0.3 sec and which increase pressure below the knee, and tends to prevent the vein to expand so that blood cannot flow downward towards the foot. The cycle can then begin again.

1.-10. (canceled)

11. A device for providing pressure to a body limb, comprising:
(a) an actuating unit configured to apply a varying pressure to a first region of the limb;
(b) a sensing unit configured to monitor a state of a second region of the limb and generating a time dependent signal indicative of the state of the second region of the limb; and
(c) a control unit comprising a processor configured to
receive the signal generated by the sensing unit,
analyze the signal to determine when the second region of the limb is in muscle vein pump diastole, and
activate the actuating unit to generate a time dependent pressure on the first region of the body limb when the limb is in muscle vein pump diastole.

12. The device according to claim 11, wherein the control unit is further configured to reduce pressure applied to the first region of the limb when the first region of the limb is in muscle vein pump systole.

13. The device according to claim 11, wherein the sensing unit senses a circumference or a movement of the second region of the limb

14. The device according to claim 13, wherein the sensing unit comprises an elastic band and a transducer monitoring an extent of stretching of the elastic band.

15. The device according to claim 13, wherein the sensing unit senses electrical activity of a muscle in the limb.

16. The device according to claim 11, wherein the actuating unit comprises a band or ring configured to surround the first region of the limb.

17. The device according to claim 11, wherein the actuating unit comprises an inflatable ring configured to surround the first region of the limb.

18. A method for providing pressure to a body limb, comprising:
(a) monitoring a state of a second region of the limb and generating a time dependent signal indicative of the state of the second region of the limb;
(b) analyzing the signal to determine when the second region of the limb is in muscle vein pump diastole; and
(c) generating a time dependent pressure on the first region of the body limb when the limb is in muscle vein pump diastole.

19. The method according to claim 18, further comprising reducing pressure applied to the first region of the limb when the first region of the limb is in muscle vein pump systole.

20. The method according to claim 18, wherein the step of monitoring the state of the second region of the limb comprises sensing a circumference or a movement of the second region of the limb.

21. The method according to claim 20, wherein the step of sensing a circumference or a movement of the second region of the limb unit comprises monitoring an extent of stretching of an elastic band surrounding the limb.

22. The method according to claim 18, wherein the step of monitoring the state of the second region of the limb comprises sensing electrical activity of a muscle in the limb.

23. The method according to claim 18, wherein the step of generating a time dependent pressure on the first region of the body limb comprises constricting a band or ring surrounding the first region of the limb.

24. The method according to claim 23, wherein the step of generating a time dependent pressure on the first region of the body limb comprises inflating an inflatable ring surrounding the first region of the limb.
